(19) United States
(12) Patent Application Publication
(10) Pub. No.: US 2017/0135647 A1
(43) Pub. Date: May 18, 2017

(54) Systems Using Fingerprint images as Diagnostic Detection systems for Type 2 Diabetes

(71) Applicant: Ohio University, Athens, OH (US)

(72) Inventors: Molly R. Morris, Athens, OH (US); Bjoern C. Ludwar, Athens, OH (US)

(73) Assignee: Ohio University, Athens, OH (US)

(21) Appl. No.: 15/318,142

(22) PCT Filed: Jun. 23, 2015
(86) PCT No.: PCT/US15/37121

§ 371 Date: Dec. 12, 2016

Related U.S. Application Data
(60) Provisional application No. 62/018,164, filed on Jun. 27, 2014.

(51) Int. Cl. A61B 5/00; A61B 5/1172; A61B 5/107

(52) U.S. Cl. CPC A61B 5/7275; A61B 5/1079; A61B 5/1172; A61B 2503/02; A61B 2503/04

(57) ABSTRACT

Method and kits for determining a propensity to develop Type 2 diabetes mellitus (T2DM) in an individual by measuring an asymmetry of a captured fingerprint from the individual are described.

### CROSS-REFERENCE TO RELATED APPLICATIONS

This application claims the priority to U.S. Provisional Application No. 62/018,164, filed Jun. 27, 2014, the entire disclosures of which are expressly incorporated herein by reference.

### STATEMENT REGARDING FEDERALLY FUNDED SPONSORED RESEARCH

This invention not was made with any government support and the government has no rights in the invention.

### BACKGROUND OF THE INVENTION

Type 2 diabetes mellitus (T2DM) is the most common form of diabetes, affecting nearly 26 million US adults aged 20 years or older, with 1.5 million more adult cases diagnosed each year, as noted by Centers for Disease Control and Prevention in 2011. The CDC predicts that 1 in 3 Americans born in 2000 will develop diabetes in their lifetime. While millions of Americans have been diagnosed with T2DM, a serious problem is that many more are unaware they are at high risk. One-third to one-half of people with T2DM are left undiagnosed and, hence, untreated. It has been shown that aggressive lifestyle intervention can delay or prevent T2DM in those at high risk. It is also believed that earlier diagnosis and treatment can prevent or delay the serious complications related to the disease and improve health outcomes. Since one third of people have a complication from T2DM at the time they are diagnosed and duration of hyperglycemia is directly related to complications, the earlier diagnosis and intervention could have a significant impact on complication prevention.

With an 11% per year progression from pre-diabetes to T2DM there is clear a lag from conversion to diagnosis. However, the benefits of implementing preventive measures requires identifying those at risk before they develop T2DM, as 30-50% of individuals with newly diagnosed T2DM have already developed complications at the time of diagnosis.

T2DM is largely preventable and wholly treatable. A healthy lifestyle is very effective at preventing T2DM. Implementing preventive measures will be more successful once those at risk are identified. The currently available models for determining a risk of developing T2DM are based on several different factors, including being overweight, particularly if your body stores fat primarily in your abdomen, body mass index (BMI), waist circumference, history of high glucose levels, inactivity, family history, race, age (although T2DM is increasing dramatically among children, adolescents and younger adults), pre-diabetes, and gestational diabetes. However, all of these models are sometimes inadequate to indentify everyone at risk.

In addition, T2DM is a disease with multiple genetic and environmental influences, making it difficult to determine any given individual's risk. If an individual knows they are at risk for T2DM, they can take necessary preventive measures to avoid or delay developing this disease and further prevent the complications of this disease.

Since T2DM is a disease with multiple genetic and environmental influences, such that even DNA sequencing of an individual's entire genome may not provide as predictive power as an indicator of an individual's growth strategy. Therefore, there is a great need for an inexpensive but comprehensive screening detection system for diabetes in humans.

In spite of considerable research into therapies to diagnose and treat this disease, it remains difficult to treat effectively, and the mortality observed in patients indicates that improvements are needed in the diagnosis, treatment and prevention of this disease.

There is no admission that the background art disclosed in this section legally constitutes prior art.

### SUMMARY OF THE INVENTION

In a first broad aspect, there is described herein a detection system to diagnose Type 2 diabetes mellitus (T2DM) using the incidence of fluctuating asymmetry (FA) between homologous in fingerprints (e.g., the first finger on the right hand has a different number of ridges in the print than the first finger on the left hand). The degree of asymmetry is significantly greater in individuals with T2DM.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1A: Two loop fingerprints (Li and L2) and one whorl patterned fingerprint (R1) with vectors (black lines shown) for counting ridge counts (rc) using pattern analyses.

FIG. 1B: For the wavelet analyses, image of the prints are first cropped around the core point to a size of 64×64 pixels.

FIG. 1C: Each cropped image is then divided into four quadrants, and three levels of Haar wavelet decomposition are computed for each quadrant to obtain the transformed image shown.

FIG. 1D: Differences between prints (measure of symmetry) using both methods are shown. Pattern analysis ridge count difference (Arc), and wavelet based methods Euclidian distance 1×1 of the feature vectors. The feature vectors are generated from the transformed image and consist of the standard deviations for each of the 4×3×3=36 high frequency decompositions. This feature vector is considered an overall description of the individual fingerprint. Note that the traditional ridge count assigns very high numbers to whorl patterns. (R1=27.5) despite the ridges actually being not very dense. The wavelet-based method better reflects the density of a print; R1 is more similar to L2 (somewhat less dense, 1×1=317.7) than L (very dense, 1×1=343.7).

FIG. 2 is a graph showing the repeatability of ridge counts (rc).

FIG. 3 is a graph showing wavelet score arc and ABS differences in ridge count for score for T2DM and controls.

### DETAILED DESCRIPTION OF THE PREFERRED EMBODIMENT(S)

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### DEFINITIONS

In order to facilitate review of the various embodiments of the disclosure, the following explanations of specific terms are provided:

Dermatoglyphics: The scientific study of fingerprints.

Directional Asymmetry: When a bilateral trait deviates from symmetry, and is more often larger on one side as compared to the other.

Fluctuating Asymmetry: The deviation from perfect bilateral symmetry that is directionally random.

Genome: The entirety of an organism's hereditary information.

Gestational diabetes: A condition in which the glucose regulation and resulting levels become abnormal while a woman is pregnant (assumption of normality prior to pregnancy).

Gestational stages: Timing of the development of a fetus during pregnancy.

Homologous finger:/fingerprint: The matching finger on the other hand (e.g., ring finger on right hand is homologous to the ring finger on left hand).

Leptokurtotic: A distribution with positive excess kurtosis, or with the tails of the distribution being very large.

Mesenchyme: A type of undifferentiated loose connective tissue.

Prediabetes: A condition of abnormal glucose regulations (impaired fasting glucose or impaired glucose tolerance) that is above normal but not diagnostic of diabetes.

Ridge Pattern: Friction ridge patterns are commonly described as one of three patterns: arch (˜5% of fingers), whorl (˜30-35% of fingers) or loop (˜60-65% of fingers).

Rolled Fingerprint: Impression of a single fingerprint taken by rolling the finger from one side of the nail to the opposing side of the nail.

Slap Fingerprint: Flat impression of the central part of fingerprints taken by typically pressing four fingers against a scanner or fingerprint card. Thumbs are typically printed separately.

Standard Deviation: The square root of the variance of a measure, and is used to express the variability of a population.

Type 1 diabetes (TIDM): Formerly termed Insulin-Dependent DM (IDDM) or juvenile diabetes). An autoimmune condition in which the insulin producing beta cells of the pancreas are destroyed, resulting in an individual not secreting sufficient insulin and therefore needing exogenous insulin to live.

Type 2 diabetes (T2DM): Formerly termed Non-Insulin-Dependent DM (NIDDM) or adult-onset diabetes). A condition that results from genetic abnormalities combined with environmental and lifestyle risks that results in abnormal glucose values that result from insulin resistance, abnormal glucose production from the liver, or impaired insulin secretion.

Volar Pads: Transient swellings of the mesenchyme under the epidermis on the palmar surface of the hands and soles of the feet of the human fetus. The size, height and shape are thought to determine the friction ridge pattern type and count of fingerprints.

Therapeutic: A generic term that includes both diagnosis and treatment. It will be appreciated that in these methods the “therapy” may be any therapy for treating a disease including, but not limited to, pharmaceutical compositions, gene therapy and biologic therapy such as the administering of antibodies and chemokines. Thus, the methods described herein may be used to evaluate a patient before, during and after therapy, for example, to evaluate the reduction in disease state.

Adjunctive therapy: A treatment used in combination with a primary treatment to improve the effects of the primary treatment.

Clinical outcome: Refers to the health status of a patient following treatment for a disease or disorder or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Decrease in survival: As used herein, “decrease in survival” refers to a decrease in the length of time before death of a patient, or an increase in the risk of death for the patient.

Patient: As used herein, the term “patient” includes human and non-human animals. The preferred patient for treatment is a human. “Patient,” “individual” and “subject” are used interchangeably herein.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Poor prognosis: Generally refers to a decrease in survival, or in other words, an increase in risk of death or a decrease in the time until death. Poor prognosis can also refer to an increase in severity of the disease, such as an increase in spread (metastasis) of the cancer to other tissues and/or organs.

Screening: As used herein, “screening” refers to the process used to evaluate and identify candidate agents that affect such disease.

General Description

Described herein is an easily useable screening or detection system that identifies individuals predisposed to develop T2DM.

In a first broad aspect, there is provided herein a method for predicting a likelihood of developing T2DM in an individual by:

detecting at least one asymmetry in homologous fingerprint images taken from an individual;

assigning a risk score to the asymmetry detected; and

predicting the likelihood of developing T2DM when the asymmetry score is assigned a high risk score; and/or

predicting a less likely chance of developing T2DM when the asymmetry score is assigned a low risk score.

In certain embodiments, the test expression level is determined by wavelet analysis of specific features from the fingerprint images.

In certain embodiments, the method further comprises designing a treatment plan based on the diagnosis. In certain embodiments, the method further comprises administration of a treatment based on the diagnosis. In certain embodiments, the method further comprises determining prognosis based on the diagnosis.

In one embodiment, there is described herein a system method for determining a propensity to develop Type 2 diabetes mellitus (T2DM) in an individual. The system includes:

a) measuring an asymmetry between captured fingerprint images from homologous fingers of the individual by using wavelet analysis to determine a degree of a fluctuating asymmetry;

b) determining that an elevated amount of asymmetry measured in step (a) relative to the amount of asymmetry in a control sample shows the propensity to develop T2DM by setting a boundary value between a degree of asymmetry in homologous fingerprint images collected from a control population and a degree of asymmetry in the homologous fingerprint image collected from the individual as an evaluation criterion, and

c) determining the risk for developing T2DM being relatively high in a case where the degree of asymmetry of the homologous fingerprint image measured is high as compared to a control.

In another embodiment, there is described herein a method for determining whether an individual has Type 2 diabetes mellitus (T2DM) or a pre-disposition for developing T2DM, wherein the method comprises the steps of:

determining the presence or absence of asymmetry in homologous fingerprint images taken from the individual, and based on the presence or absence of such asymmetry; and,

determining whether the individual has T2DM or a pre-disposition for developing T2DM, and, optionally, recommending a particular treatment for T2MD or pre-T2DM condition.

In another embodiment, there is described herein a method of diagnosing whether an individual has, or is at risk for developing, Type 2 diabetes mellitus (T2DM), comprising:

receiving at least one set of homologous fingerprint images extracted from the individual;

measuring by wavelet analysis a level of asymmetry between the set of homologous fingerprint images;

comparing the level of asymmetry between the set of homologous fingerprint images of the individual to a control level of symmetry in normal fingerprint images; and

diagnosing whether the individual has, or is at risk for developing, T2DM if the level of asymmetry between the homologous fingerprint images in the set from the individual is greater than the level of asymmetry in the corresponding control.

In certain embodiments, the asymmetry is measured during gestation of the individual. Also, in certain embodiments, the method further includes determining a point in time during gestation that the individual is most susceptible to environmental stressors that interact with the genes for diabetes. The method can also further include indicating when during gestation a therapeutic intervention aimed at decreasing the incidence of diabetes is beneficial. For example, the environmental stressor can be a mother's diabetes.

In certain embodiments, a first homologous fingerprint image is compared with a second homologous fingerprint image by calculating Euclidean or Manhattan distances between the first and second homologous fingerprint images.

In another embodiment, there is described herein a method for determining whether or not an individual has increased risk of type 2 diabetes mellitus (T2DM), comprising:

obtaining least one set of homologous fingerprint images from the individual;

conducting laboratory analysis of the sample so as to obtain symmetry data of the homologous fingerprint images, wherein the laboratory analysis is wavelet analysis; and

determining that the individual has increased risk of T2DM if the asymmetry data indicate that the set of homologous fingerprint images are more asymmetrical than a control; or

determining that the individual has no increased risk of T2DM if the asymmetry data indicate that the set of homologous fingerprint images are not more asymmetrical than the control.

In certain embodiments, the method further includes the step of correlating the data with similar data from a reference population.

In another embodiment, there is provided herein a method of treating an individual having type 2 diabetes mellitus (T2DM) comprising:

measuring the expression of at least one set of homologous fingerprint images from the individual;

comparing the asymmetry between homologous fingerprint images in the set of homologous fingerprint biomarker to a corresponding control;

determining whether the asymmetry is high; administering at least one therapeutic treatment if the asymmetry is high, in an amount sufficient to modulate symptoms associated with T2DM, wherein the symptoms of T2DM are decreased after administration, thereby treating the subject.

Also described herein is a medium for holding instructions for performing a method for determining whether an individual has T2MD or a pre-disposition for developing T2DM.

Also described herein is an electronic system for use in determining whether an individual has T2MD or a pre-disposition for developing T2DM.

In certain embodiments, the medium and/or electronic system is configured for receiving information associated with the individual and/or acquiring from a network such information associated with the individual.

A method for determining whether an individual has T2DM or a pre-disposition for developing T2DM associated with asymmetry in one or more homologous fingerprints, the method comprising the steps of:

receiving information associated with the homologous fingerprints,

receiving phenotypic information associated with the individual,

acquiring information from the network corresponding to the homologous fingerprints and/or T2DM, and based on one or more of the phenotypic information, the homologous fingerprints, and the acquired information, and

determining whether the individual has T2DM or a pre-disposition for developing T2DM, and, optionally,

further comprising the step of recommending a particular treatment for the T2DM or pre-T2DM disease condition.

Also described herein is a kit useful or determining the whether an individual as has, or is at risk for developing type 2 diabetes mellitus (T2DM). the kit can include a device for obtaining at least one image of at least one fingerprint of at least one finger of the individual, and for comparing the at least one obtained fingerprint to a control sample set using wavelet analysis; and instructions for the use of the fingerprint image in determining the diagnosis of T2DM or risk of developing T2DM, wherein the instructions comprise providing directions to compare the wavelet analysis of the fingerprint image to a control.

Also described herein is a kit for the assessment of a clinical condition of an individual, comprising: one or more devices for obtaining a predetermined fingerprint image from the individual, and instructions for determining fluctuating asymmetry in the fingerprint image.

In certain embodiments, the fingerprint images are laid down in a database, such as an internet database, a centralized or a decentralized database.

In particular aspects, described herein is a detection system where fluctuating asymmetry (FA) in homologous fingerprints is used to identify individuals with the propensity to develop T2DM.

The system herein provides advantages over methods that relied solely on ridge counts to detect differences in symmetry. Furthermore, different ridge patterns and the limited variation in ridge counts (typical range: 2 to 20) reduce the potential sensitivity of the analysis and increase the impact of an error during the somewhat subjective counting procedure. In contrast, the system herein uses wavelet based analysis method, either alone, or in parallel with traditional ridge counts, in order to avoid these limitations.

Additionally, the comparison of the asymmetry across different homologous fingers provides valuable information regarding the timing of gestational environmental influences, which also leads to a better indication of when screening for gestational diabetes would be most valuable.

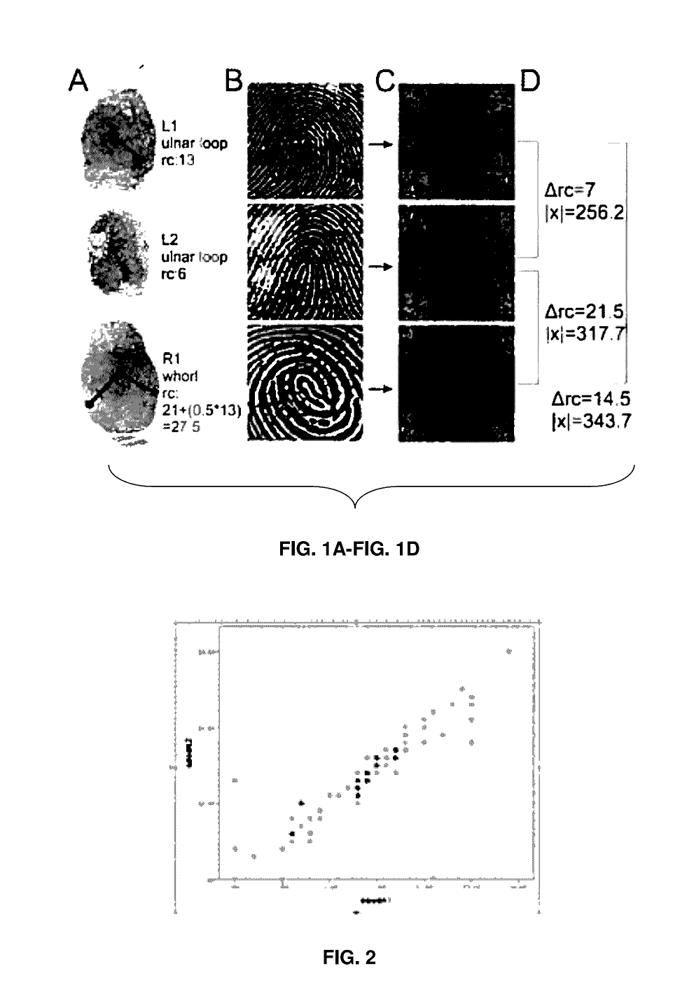

Pattern analysis can only compare the fingerprint pattern (simplest classification includes arch, loop or whorl) or generates ridge counts that are then compared between prints, as shown in FIG. 1). It is to be noted that there are severe limitations to using only pattern analysis, as pattern analysis is a very coarse measurement (using only about 30 different values), and depending on the fingerprint image, the count can be fairly subjective and easily vary by ±2.

In contrast, in the present T2DM detection system, a wavelet-based analysis is used which measures different features; yet still provides a less complex description of the fingerprint (e.g., a feature vector of 36 numbers, shown in FIG. 1A-FIG. 1D) which can then be compared with a second print by calculating the Euclidean or Manhattan distance. The wavelet-based method has several advantages: ability to detect overall differences, is mostly immune to variation in acquisition (i.e., finger placement on the scanner), and provides a similarity score that can be used as a score of symmetry.

In one embodiment, to assess FA in fingerprints as a risk score for T2DM, receiver operating characteristic (ROC) curves can be created for each finger, in which the true-positive rate and the false-positive rate are paired across all potential cutoff points that distinguished between individuals with and without T2DM. In the ROC curve, the true-positive rate (sensitivity) is the proportion of individuals with T2DM that the FA correctly predicted as having T2DM; the higher the rate, the more accurate the test. The false-positive (91=specificity) is the proportion of the control individuals that the FA score incorrectly predicted as have T2DM; the lower the rate, the more accurate the test. The true-negative rate (specificity) is therefore the proportion of the controls that FA correctly classified as not having T2DM. It is desired that the diagnostic detection system have a high true-positive rate and a low false-positive rate in partitioning individuals with, and without, T2DM.

Test for T2DM Susceptibility

In addition, the diagnostic detection system described herein can be useful to determine when during gestation embryos are most susceptible to environmental stressors (e.g., mother with diabetes) that interact with the genes for diabetes, indicating when during gestation therapeutic intervention aimed at decreasing the incidence of diabetes must be instituted. It is now believed that asymmetry in bilateral traits can be a strong indicator of a genotype that optimizes growth over development. In addition, while not wishing to be bound by theory, it may be that plasticity is superior to discrete alternative reproductive tactics, and demonstrates the importance of identifying alternative growth strategies within a species.

Fingerprint Images

In certain embodiments, to obtain a print showing all friction ridges of an individual finger, a rolled fingerprint image can be used. While this rolled fingerprint image is more time consuming than acquiring slap fingerprint images, use of a rolled fingerprint image can ensure a precise classification of the ridge pattern and an accurate ridge count. For example, the fingerprint images of all fingers on both hands can be stored as uncompressed digital images on a laptop dedicated to the project with an encrypted storage device.

Fingerprints were collected using the Crossmatch Verifier 320 scanner. The prints of fingers on both hands were stored as uncompressed digital images on a laptop dedicated to the project with an encrypted storage device. Fingerprints were scored for similarity between homologous fingers (symmetry) using both ridge counts (pattern analysis) and wavelet based methods.

Data

The prints from 101 individuals from this cohort (out of 240 collected) have been scored for differences in ridge counts and Haar wavelet decomposition (similarity scores). The results show that asymmetry in fingerprints is an indicator of propensity to develop diabetes. Described herein are the analyses of the wavelet decomposition scoring, as compared to pattern analysis.

First, to consider the predictability of asymmetry in fingerprints for developing diabetes use multivariable logistic regressions can be used. In the model for finger 1 (thumb), the significant independent predictors of diabetes state (T2DM or control) were age (Wald X²=24.95, P=0.0001) and asymmetry score (Wald X²=5.62, P=0.018). Sex was not significant (P=0.545). This analysis shows that an increase in one point for the asymmetry score for finger 1 (cohort mean=227.7, SE=83.2) increases the odds of being T2DM by a multiplicative factor of 1.0 (Exp(B)=1.012).

Second, to consider the predictability of asymmetry in fingerprints to provide additional information about environmental changes during gestation, certain variables (e.g. age, sex, ethnicity, and the like) were measured. In particular, variations in symmetry scores for each set of homologous fingerprint images were determined using General Linear Models. For example, in the model for finger 3 (middle finger), age and sex significantly influenced variation in asymmetry scores (age, F=8.381, P=0.005; sex F=5.588, P=0.020). Based on the belief that asymmetry is due to a tradeoff between growth and development), the results from finger 3 show that males grow faster than females during the time the prints for finger 3 were forming.

Additional Data Analysis

Fingerprints do not change throughout an individual's lifetime; therefore the diagnostic detection system described herein is also useful for predicting risk of developing diabetes at any postnatal age and prior to development of any phenotypic correlates with diabetes.

Also, in certain embodiments, one or more additional variables can be co-determined and used with the detection system described herein. As such, the control data can specifically include older individuals without diabetes and/or older individuals without diabetes that have healthy lifestyles. It is to be noted that it is more difficult to obtain readable fingerprints from older adults. Therefore, scanners with higher resolution (Dermalog LF10, Optical Resolution 500 ppi; Dynamic Range Greater than 8 bit, yielding over 256 gray scale depth) can be useful in order to obtain the fingerprints.

Data Analysis

Receiver operating characteristic (ROC) curves for each finger are created, in which the true-positive rate (sensitivity) and the false-positive rate (1−specificity) are paired across all potential cutoff points that distinguished between individuals with and without T2DM. In certain embodiments, the area under curve of 0.8, or greater, is predictive at any age.

In certain embodiments, the diagnostic detection system can be used in concert with genomic sequencing that has identified genes associated with T2DM. While not wishing to be bound by theory, it is now believed that genomic sequencing will have more false positives than the fingerprint detection system described herein because possessing the genes for T2DM may be necessary, but not sufficient for, the development of diabetes. Thus, a comparison between the two methods not only supports fingerprint asymmetry as a powerful new detection system for detecting risk, but also shows that gene×environment interactions (which fingerprints can detect) are important when explaining variation in the propensity to develop T2DM across humans.

### Examples

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

Materials and Methods

Individuals were seen either at the UMA Diabetes/Endocrine Center or the UMA Family Medicine Center in Athens Ohio. Individuals were included in the control group if they had no individual or family history of diabetes or any insulin resistant syndrome (such as polycystic ovarian syndrome, metabolic syndrome). This was confirmed clinically by a clinical interview/exam by the Diabetes Endocrine Center faculty or a detailed chart review. The individual's medical records were used to exclude those individuals with chromosomal syndromes (e.g., Down, Turner, Klinefelter), monogenic disease (e.g., cystic fibrosis, MODY), polygenic morbidity (e.g., cleft palate and cleft lip with or without cleft palate), and females suffering from endometrial carcinoma or carcinoma of cervix, all of which are correlated with fingerprint asymmetries. Fingerprints were collected using the Crossmatch Verifier 320 scanner. The fingerprints of fingers on both hands were stored as uncompressed digital images on a laptop dedicated to the project with an encrypted storage device.

Fingerprints were scored for similarity between homologous fingers (symmetry) using both ridge counts (pattern analysis) and wavelet based methods.

FIG. 2 is a graph showing the repeatability of ridge counts (rc).

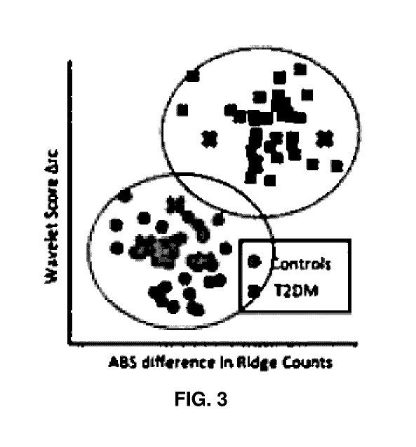

FIG. 3 is a graph showing wavelet score arc and ABS differences in ridge count for score for T2DM and controls.

Ridge Counts (Pattern Analysis)

Individuals were removed from the study that did not have a complete set of prints. Sample Information: N=85 females, N=51 males, N=44 Controls, N=62 T2DM, and N=21 T1DM. Data was normalized using the following transformation: LN (score+0.333)

Both sex and diabetes state (T2 or Control) were significant in the Generalized Linear model which analyzed variation in the ridge count asymmetry scores for finger 4.

Males were more asymmetrical than females (t=2.07, P=0.04)

Individuals with T2DM were more asymmetrical than controls (t=−3.13, P=0.002).

Finger 4 was the only finger where diabetes state (T2DM or Control) influenced variation in ridge count asymmetry scores. There was no difference in FA between T1DM individuals and Controls for any of the fingers, likely due to the small sample size.

Wavelet Analysis

It is to be noted that many more of the prints could be analyzed using wavelet analysis than the ridge count analysis, as the wavelet analysis method is less reliant on getting a clear print. However, there appeared to be a bimodal distribution in this data set, with a set of data points under 40 that were separate from the rest of the data (FA plotted by age).

Sample information: Controls N=82 (Average Age=31.1707, SD=16.815), T2DM N=196 (Average Age=59.44, SD=13.421), and T1DM N=52 (Average Age=42.3, SD=16.397).

Age, diabetes state (T2DM or Control), gender, and an interaction between age and diabetes state were all significant in a Generalized Linear model explaining variation in the asymmetry scores for finger 4.

Age: There was a negative correlation between age and symmetry.

Gender: Males were more asymmetrical than females.

Diabetes State and Age Interaction: There was a significant difference in the relationship between diabetes state and age depending on whether individuals were under the age of 40 as compared to over the age of 40: ‘Under the age of 40’=Controls were more asymmetrical than T2; ‘Over the age of 40’=Controls were more symmetrical than T2.

While there was no relationship with asymmetry for individuals under 40, it is to be noted that for ‘Over 40’ the T1DM were more asymmetrical than controls (i.e., the same pattern as was detected for T2DM). When the FA was compared between the T1DM and the T2DM, it is noted that TIDM were significantly more asymmetrical for the individuals ‘Under 40,’ but there was no significant difference between T1DM and T2DM across individuals ‘Over 40.’

Individuals with T2DM were in most cases more asymmetrical, controlling for age. However, it was noted that, using the wavelet detection system described herein that, for individuals ‘Under 40’ the controls were more asymmetrical.

Also, in using the wavelet detection system described herein, the FA in individuals with Type 1 Diabetes (T1DM) also presented asymmetry scores similar to what was detected in individuals with T2DM.

Gestational Development and T2DM

Fingerprints are influenced by an interaction between genetics and the inter-uterine environment. Once formed, however, fingerprints do not change over an individual's lifetime.

There is uncertainty about precisely when during gestation the environment impacts the metabolism of the adult. As there are preventive measures that could be taken during pregnancy, fingerprints could help to more clearly identify the timing of this critical period during gestation.

To determine that some stages of gestational development are more important in the onset of T2DM, the degree of FA of the different fingers can be compared.

Thus, in certain embodiments, such detection system is useful to identify the gestational stages when the uterine environment is more likely to influence an individual's propensity to develop diabetes. While not wishing to be bound by theory, it is now believed that the ridge pattern of fingerprints results from stress and tension lines in volar pads. These volar pads are transitional swellings of the embryo's hand mesenchyme that begin 7-8 weeks into gestation. Interestingly, volar pad development begins at the thumb and progresses towards the little finger, allowing developmental deficiencies to arise during different times of gestation to present as asymmetry for different fingers. Volar pad size is determined by many factors, among them diet of the mother and other factors that affect growth rate. This suggests volar pads are influenced by the same environmental factors that influence diabetes. At 10-10.5 weeks estimated gestational age (EGA) primary ridges form through rapid division of epidermal cells. This is possibly correlated with or even triggered by innervation from spinal cord levels C6, C7, and C8. Each of these spinal cord levels innervates different fingers. By 16 weeks EGA, the tissue surrounding the volar pads has caught up in growth and the originally enlarged volar pads now blend in with the finger contours. The timing (determined by growth rate) of this process influences the ridge pattern. It is now believed that volar pad height, size, and shape determine the friction ridge shape by influencing the stress across the skin. High volar pads result in high ridge counts and whorl patterns, low pads to low counts and arch patterns.

In addition, environmental stress during gestation can influence fingerprint ridge counts. Environmental influences during gestation are related to risk for T2DM as well, and can affect subsequent adult metabolic rates as well as adult body size. A baby of reduced birth size, for example, carries an increased risk of insulin resistance in later life. Still, there is uncertainty about precisely when during gestation the environment impacts the metabolism of the adult. As there are preventive measures that could be taken during pregnancy, more clearly identifying the timing of this critical period during gestation can be very important.

By comparing different aspects of fingerprints ridge count and shape across fingers, it is now possible to determine when during gestation the environment was unfavorable, leading to diabetics for those with specific genes.

In certain embodiments, the method further comprises wherein the fingerprint images are obtained from the fetus over time.

Some stages of gestational development appear to be more important in the onset of T2DM. When the degree of FA of the different fingers were compared to determine which finger was the most predictive of DM, only finger 4 was predictive.

There was difference between males and females in the asymmetry scores. Males were more asymmetric than females for finger 4 for wavelet analysis. The pattern of males being more asymmetrical was the same in the ridge count data but not significant.

Electronic Apparatus Readable Media, Systems, Arrays and Methods of Using the Same

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

The system can also include detection apparatus that is used to detect the desired information, using any of the approaches noted herein. For example, a detector configured to obtain and store fingerprint images or a fingerprint reader can be incorporated into the system. Optionally, an operable linkage between the detector and a computer that comprises the system instructions noted above is provided, allowing for automatic input of specific information to the computer, which can, e.g., store the database information and/or execute the system instructions to compare the detected specific information to the look up table.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Sequel™, Oracle™, Paradox™) can be adapted to the present invention by inputting a character string corresponding to an allele herein, or an association between an allele and a phenotype. For example, the systems can include software having the appropriate character string information, e.g., used in conjunction with a user interface (e.g., a GUI in a standard operating system such as a Windows, Macintosh or LINUX system) to manipulate strings of characters. Specialized sequence alignment programs such as BLAST can also be incorporated into the systems of the invention for alignment of nucleic acids or proteins (or corresponding character strings) e.g., for identifying and relating multiple alleles.

As noted, systems can include a computer with an appropriate database and an allele sequence or correlation of the invention. Software for aligning sequences, as well as data sets entered into the software system comprising any of the sequences herein can be a feature of the invention. The computer can be, e.g., a PC (Intel x86 or Pentium chip-compatible DOS™ OS2™ WINDOWS™ WINDOWS NT™, WINDOWS95™, WINDOWS98™, WINDOWS2000, WINDOWSME, or LINUX based machine, a MACINTOSH™, Power PC, or a UNIX based (e.g., SUN™ work station or LINUX based machine) or other commercially common computer which is known to one of skill. Software for entering and aligning or otherwise manipulating sequences is available, e.g., BLASTP and BLASTN, or can easily be constructed by one of skill using a standard programming language such as Visualbasic, Fortran, Basic, Java, or the like.

In certain embodiments, the computer readable medium includes at least a second reference profile that represents a level of at least one additional fingerprint asymmetry score from one or more samples from one or more individuals exhibiting indicia of T2DM.

In another aspect, there is provided herein a computer system for determining whether an individual has, is predisposed to having, T2DM, comprising a database and a server comprising a computer-executable code for causing the computer to receive a profile of an individual, identify from the database a matching reference profile that is diagnostically relevant to the individual profile, and generate an indication of whether the individual has, or is predisposed to having, T2DM.

In another aspect, there is provided herein a computer-assisted method for evaluating the presence, absence, nature or extent of T2DM in an individual, comprising: i) providing a computer comprising a model or algorithm for classifying data from a sample obtained from the individual, wherein the classification includes analyzing the data for the presence, absence or amount of at least asymmetry homologous fingerprint score; ii) inputting data from the fingerprint image sample obtained from the individual; and, iii) classifying the biological sample to indicate the presence, absence, nature or extent of T2DM.

As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; and general hard disks and hybrids of these categories such as magnetic/optical storage media. The medium is adapted or configured for having recorded thereon a marker as described herein.

As used herein, the term “electronic apparatus” is intended to include any suitable computing or processing apparatus or other device configured or adapted for storing data or information. Examples of electronic apparatus suitable for use with embodiments of the present invention include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems.

As used herein, “recorded” refers to a process for storing or encoding information on the electronic apparatus readable medium. Those skilled in the art can readily adopt any method for recording information on media to generate materials comprising the markers described herein.

A variety of software programs and formats can be used to store the fingerprint image information on the electronic apparatus readable medium. Any number of data processor structuring formats (e.g., text file or database) may be employed in order to obtain or create a medium having recorded thereon the markers. By providing the markers in readable form, one can routinely access the information for a variety of purposes. For example, one skilled in the art can use the information in readable form to compare a sample fingerprint image with the control information stored within the data storage means.

Thus, there is also provided herein a medium for holding instructions for performing a method for determining whether an individual has T2MD or a pre-disposition for developing T2DM, wherein the method comprises the steps of determining the presence or absence of asymmetry in homologous fingerprints, and based on the presence or absence of such asymmetry, determining whether the individual has T2DM or a pre-disposition for developing T2DM, and/or recommending a particular treatment for T2MD or pre-T2DM condition. It is contemplated that different entities may perform steps of the contemplated methods and that one or more means for electronic communication may be employed to store and transmit the data. It is contemplated that raw data, processed data, diagnosis, and/or prognosis would be communicated between entities which may include one or more of: a primary care physician, patient, specialist, insurance provider, foundation, hospital, database, counselor, therapist, pharmacist, and government.

There is also provided herein an electronic system and/or in a network, a method for determining whether an individual has T2MD or a pre-disposition for developing T2DM, wherein the method comprises the steps of determining the presence or absence of asymmetry in homologous fingerprints, and based on the presence or absence of such asymmetry, determining whether the individual has T2DM or a pre-disposition for developing T2DM, and/or recommending a particular treatment for T2MD or pre-T2DM condition. The method may further comprise the step of receiving information associated with the individual and/or acquiring from a network such information associated with the individual.

Also provided herein is a network, a method for determining whether an individual has T2DM or a pre-disposition for developing T2DM associated with asymmetry in one or more homologous fingerprints, the method comprising the steps of receiving information associated with the homologous fingerprints, receiving phenotypic information associated with the individual, acquiring information from the network corresponding to the homologous fingerprints and/or T2DM, and based on one or more of the phenotypic information, the homologous fingerprints, and the acquired information, determining whether the individual has T2DM or a pre-disposition for developing T2DM. The method may further comprise the step of recommending a particular treatment for the T2DM or pre-T2DM disease condition.

There is also provided herein a business method for determining whether an individual has T2DM or a pre-disposition for developing T2DM, the method comprising the steps of receiving information associated with fingerprint images of homologous fingerprints, receiving phenotypic information associated with the individual, acquiring information from the network corresponding to the homologous fingerprints and/or T2DM, and based on one or more of the phenotypic information, the homologous fingerprints, and the acquired information, determining whether the individual has T2DM or a pre-disposition for developing T2DM. The method may further comprise the step of recommending a particular treatment for T2DM or pre-T2DM condition.

Kits

Particular embodiments are directed to kits useful for the practice of one or more of the methods described herein. Kits for using detection method described herein for therapeutic, prognostic, or diagnostic applications and such uses are contemplated by the inventors herein. The kits can include devices for capturing fingerprint images, as well as information regarding a standard or normalized profile or control.

Also, the kits can generally comprise, in suitable means, distinct containers or image collecting devices for each individual fingerprint image. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of the fingerprint images and/or data derived therefrom.

The kits described herein can reduce the costs and time associated collecting a variety of images. The kits may be used by research and commercial laboratories and medical end users to facilitate collection of fingerprint data in remote locations.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

1. (canceled)

2. A method for determining a propensity to develop Type 2 diabetes mellitus (T2DM) in an individual, comprising:
a) measuring an asymmetry between captured fingerprint images from homologous fingers of the individual by using wavelet analysis to determine a degree of a fluctuating asymmetry;
b) determining that an elevated amount of asymmetry measured in step (a) relative to the amount of asymmetry in a control population by setting a boundary value between a degree of asymmetry in homologous fingerprint images collected from the control population and a degree of asymmetry in the homologous fingerprint image collected from the individual as an evaluation criterion, and
c) determining the risk for developing T2DM being relatively high in a case where the degree of asymmetry of the homologous fingerprint image measured is high as compared to the control population.

3. (canceled)

4. The method of claim 2, wherein the asymmetry is measured during gestation of the individual.

5. The method of claim 4, further including determining a point in time during gestation that the individual is most susceptible to environmental stressors that interact with the genes for diabetes.

6. The method of claim 5, further including indicating when during gestation a therapeutic intervention aimed at decreasing the incidence of diabetes is beneficial.

7. The method of claim 5, wherein the environmental stressor is a mother's diabetes.

8. The method of claim 2, wherein a first homologous fingerprint image is compared with a second homologous fingerprint image by calculating Euclidean or Manhattan distances between the first and second homologous fingerprint images.

9. A method for determining whether or not an individual has increased risk of type 2 diabetes mellitus (T2DM), comprising:
obtaining least one set of homologous fingerprint images from the individual;
conducting laboratory analysis of the sample so as to obtain symmetry data of the homologous fingerprint images, wherein the laboratory analysis is wavelet analysis; and
determining that the individual has increased risk of T2DM if the asymmetry data indicate that the set of homologous fingerprint images are more asymmetrical than a control population; or
determining that the individual has no increased risk of T2DM if the asymmetry data indicate that the set of homologous fingerprint images are not more asymmetrical than the control population.

10. The method of claim 9, further comprising the step of correlating the data with similar data from the control population.

11. The method of claim 1, further comprising the step of:
administering at least one therapeutic treatment if the asymmetry is high, in an amount sufficient to modulate symptoms associated with T2DM, wherein the symptoms of T2DM are decreased after administration, thereby treating the individual.

12. (canceled)

13. (canceled)

14. (canceled)

15. A method for determining whether an individual has T2DM or a pre-disposition for developing T2DM associated with asymmetry in one or more homologous fingerprints, the method comprising the steps of:
receiving information associated with the homologous fingerprints,
receiving phenotypic information associated with the individual,
acquiring information from the network corresponding to the homologous fingerprints and/or T2DM, and based on one or more of the phenotypic information, the homologous fingerprints, and the acquired information, and
determining whether the individual has T2DM or a pre-disposition for developing T2DM, and, optionally,
further comprising the step of recommending a particular treatment for the T2DM or pre-T2DM disease condition.

16. A kit for use with the method of claim 2 in determining the whether an individual as has, or is at risk for developing type 2 diabetes mellitus (T2DM), comprising:
a device for obtaining at least one image of at least one fingerprint of at least one finger of the individual, and for comparing the at least one obtained fingerprint to the control population using wavelet analysis; and
instructions for the use of the fingerprint image in determining the diagnosis of T2DM or risk of developing T2DM,
wherein the instructions comprise providing directions to compare the wavelet analysis of the fingerprint image to the control population.

17. (canceled)

18. The kit of claim 16, wherein the fingerprint images are laid down in a database, an internet database, a centralized or a decentralized database.

19. The method of claim 2, wherein the homologous fingerprints are of the fourth finger.

20. The method of claim 9, wherein the homologous fingerprints are of the fourth finger.

21. The method of claim 15, wherein the homologous fingerprints are of the fourth finger.
